(19) United States
(12) Patent Application Publication
(10) Pub. No.: US 2022/0133773 A1
(43) Pub. Date: May 5, 2022

(54) MEDICAMENT CONTAINING MODIFIED STARCH FOR THE TREATMENT OF ANASTOMOSES

(71) Applicant: PlantTec Medical GmbH, Lüneburg (DE)

(72) Inventors: Joachim LAAS, Bad Bevensen (DE); Kai-Oliver MUTZ, Bad Bevensen (DE); Simon WEIGMANN, Lüneburg (DE)

(21) Appl. No.: 17/527,928

(22) Filed: Nov. 16, 2021

Related U.S. Application Data
(63) Continuation of application No. 16/318,607, filed on Sep. 10, 2019, now abandoned.

(30) Foreign Application Priority Data
Jul. 7, 2016 (DE) 10 2016 008 533.9

(51) Int. Cl. A61K 31/718; A61P 17/02; A61K 9/00; A61K 9/06

(52) U.S. Cl. CPC A61K 31/718; A61K 9/06; A61K 9/0014; A61P 17/02

(57) ABSTRACT

According to the invention, a medicament a) containing or consisting of carboxymethylated starch and/or b) containing or consisting of epichlorohydrin-modified starch for treating anastomoses is disclosed. A method for preparing the medicament, and a use of the medicament.

The invention relates in particular to a medicament. The invention further relates to a method for preparing a medicament, use of the disclosed medicament, a further use, and a method according to the invention.

Following resection of portions of the stomach or intestine, to maintain the respective bodily function these sections are reconstructed by so-called anastomoses, by means of a mechanical connection between two anatomical structures, in particular by sewing them together. In these anastomoses, it is desirable that rapid wound healing and growing together occur without complications; when topically applied to such anastomotic areas, the medicament according to the invention provides excellent wound healing and coalescence effects in the joined anatomical structures such as intestinal sections, with exceptionally low risk of infection.

WO 2009/091549 A1 discloses in particular a hemostatic material, which is a modified starch having a molecular weight greater than 15,000 daltons. The modified starch is biocompatible, and is absorbed after degradation by amylases and carbohydrases. For the application, methods are disclosed in which the hemostatic material is applied to the skin and internal tissue as a powder or a gel, for example, and is used for hemostasis, adhesion prevention, improved tissue healing, wound closure, and tissue bonding in open, endoscopic, laparoscopic, and laryngoscopic surgery and traumatology. An application for treating anastomoses is not disclosed.

Totally unexpectedly, it has been shown according to the invention that the medicament according to the invention is very well suited for treating anastomoses.

### BRIEF DESCRIPTION OF THE DRAWINGS

FIG. 1: Anastomotic bursting pressure seven (7) days after creation of colon anastomoses in mice.

Table 1: Anastomotic bursting pressure per group
Group Anastomotic bursting pressure

The medicament according to the invention contains or consists of carboxymethylated starch and/or contains or consists of epichlorohydrin-modified starch, since these two types of starch surprisingly give excellent results in treating anastomoses. This applies in particular for treating anastomoses in and/or on the intestine, but also for treating anastomoses of mesenchymal organs, for example and in particular bones, tendons, muscles, fat tissue, and connective tissue, wherein connective tissue is understood to mean tissue that is present between two different tissues and/or organs and joins them together; for example, tendons are the connective tissue between bones and muscles.

It is presumed that the excellent properties are based on the following mechanisms:

As a result of the modified starches mentioned above, an early body reaction is triggered, which is accompanied by the migration of macrophages, followed by fibroblasts, which as stabilizing factors are responsible for improved anastomotic healing with improved performance.

In this regard, the medicament according to the invention is also suitable for treating epithelial defects and for treating wounds.

The medicament is advantageously present in the form of a gel; in addition, the medicament according to the invention advantageously contains water and/or at least one salt, for example and in particular sodium chloride or potassium chloride. In practice it has proven beneficial for the starch to have a molecular weight of 500,000 daltons to 11,000,000 daltons, and/or for the starch (i.e., the starch particles) to have a particle diameter of 30 microns to 100 microns.

In the simplest form of the method according to the invention for preparing the medicament according to the invention, the method involves providing the above-mentioned modified starch or using water and optionally salts to form a gel.

According to the invention, the use of the medicament according to the invention for treating the indications mentioned above is also claimed.

Furthermore, the use of a) carboxymethylated starch and/or b) epichlorohydrin-modified starch for preparing a medicament for treating anastomoses is claimed, in particular when the treatments involve those mentioned above.

Lastly, a method for treating anastomoses is also claimed in which the medicament according to the invention is used, in particular when it is topically applied.

As the result of administering the medicament according to the invention, i.e., the carboxymethylated starch or the epichlorohydrin-modified starch, either as a powder or as a gel, a gel is formed which may then act locally on the anastomotic area. The presence of hydrobeads (i.e., the above-mentioned starch particles) is associated with the increased invasion of cellular components of early healing; in this sense, activated macrophages come into contact with the surfaces of the hydrobead particles quite rapidly, forming a framework, followed by other cellular components of early healing. Although the polysaccharide framework/matrix is degraded within days, an increased number of cellular components remain in the healing area, resulting in improved healing, wherein in particular an increased number of macrophages for the macrophagocytosis of bacteria, apoptopic cells, necrotic cells, cells with foreign protein, and polysaccharide surfaces are observed. In addition, an increased number of fibroblasts and other cellular components of wound healing are also observed. The presence of cells for early healing and the subsequent wound healing processes are accelerated by these factors. These cellular components form the basis for improving anastomotic healing, and also provide better resistance to increased intraluminal pressure or other mechanical stresses in the intestinal area, resulting in reduced occurrence of leaks in intestinal anastomoses.

Histological analyses in an area have shown improved submesothelial healing after one week due to application of the above-mentioned starch gel, i.e., in a form of the medicament according to the invention showing remnants (residues) of hydrobeads, macrophages and giant cells, fibroblasts, and emerging connective tissue.

The improvement in healing results from the presence of the medicament according to the invention, which activates the early cellular healing processes, i.e., the migration of macrophages, followed by fibroblasts. The latter represent stabilizing factors for improving anastomotic healing with improved performance, i.e., better resiliency against mechanical stress and reduced occurrence of anastomotic insufficiency.

The following characterizations of the starch that is used represent advantageous embodiments.

The starch according to the invention is a crosslinked, carboxymethylated starch containing approximately 20 wt % amylose and approximately 80 wt % amylopectin in a relatively constant ratio. The amylose is rather amorphous, and the amylopectin represents the crystalline portion of the starch. The modified starch is present as a salt-starch glycolate, and is generally the sodium salt of a carboxymethyl ether of the starch. The modified starch may contain up to 10 wt % salt (generally sodium chloride). The pH is between 3 and 7.5. The percentage of salt that is bound to the modified starch is between 2 wt % and 5 wt %. The modified starch is present as a white or almost white fine, free-flowing powder. The powder is very hygroscopic, and is practically insoluble in methylene chloride. It forms a translucent suspension in water. The powder is made up of irregularly shaped, oval or pear-shaped particles, 30 to 100 μm in size, or 10 to 35 μm with rounding. The surface is typically approximately 0.2 m²/g. Occasionally occurring combinations of particles are made up of two to four particles. The particles have an eccentric hilus and clearly visible concentric grooves. The particles exhibit a distinct black cross on the hilus, between intersecting Nicol prisms. Small crystals are discernible at the surface of the particles. The particles exhibit significant swelling, up to approximately 30 times their own weight, upon contact with water or salt solutions.

The synthesis of the modified starch generally takes place in two steps, in each case as a suspension: first the crosslinking, then the carboxymethylation. Since the reactions take place in the solid phase, the substitution is inhomogeneous. Outer areas of the starch are more greatly affected by the modifications than the core areas of the starch structures. The reactions are influenced by diffusion within the starch structure and accessibility to the starch structure. Amorphous areas are more easily accessible than crystalline areas, which are less strongly modified. From a chemical standpoint, both reactions are nonspecific, and, provided that diffusion inhibition or steric hindrance is present, have no preferred reaction pattern for the individual hydroxyl groups of a glucose unit. Theoretically, the degree of substitution is 3 (with substitution of all three OH groups of a glucose unit). The actual degree of substitution appears to be approximately 0.2 to 0.4; i.e., one OH group is substituted in approximately every fourth glucose unit (not taking crosslinking into account). This degree of substitution results from the values for the bound salt.

At the end of the reactions, neutralization is carried out using succinic acid, phosphoric acid, hydrochloric acid, sulfuric acid, or citric acid. The selection of the acid has an effect on inorganic and organic substances that can be detected as residues in the modified starch.

The molecular weight is typically 500,000 to 11,000,000 daltons.

A starch particle is made up of approximately 4.5×10¹⁰ to 2.3×10¹² amylose molecules and approximately 5.6×10⁷ to 1.3×10¹⁰ amylopectin molecules. The degree of crosslinking is approximately 25-45%.

Possible starch solutions use water, isotonic saline solution, or hypotonic, isotonic, or hypertonic saline solution as the liquid phase with the following cations: sodium, potassium, ammonium, magnesium, calcium, iron(II), iron(Ill), aluminum; and the following anions: fluoride, chloride, bromide, iodide, oxide, sulfide, carbonate, sulfate, phosphate, nitrate, chromate, permanganate, hexacyanoferrate(II). Ringer's solution, Ringer's acetate solution, and Ringer's lactate solution may also be used as the liquid phase.

The subject matter according to the invention is explained in the following embodiment by way of example and in a nonlimiting manner.

### Experiment 1

Effect of Improved Wound Healing for Colon Anastomoses in the Mouse Model

Objective:

Investigation of the effect of the wound healing-improving material on the healing of colon anastomoses by measuring the anastomotic bursting pressure in the mouse model

Test Object:

Wound healing-improving material

Name:

Carboxymethylated starch

Animals:

Black C57BL/6 mice supplied by Charles River Laboratories, Research Models and Services, Germany GmbH (Sulzfeld, Germany)

Animal certificate: 84-02.04.2014.A127

A total of 23 mice (n=10 per group plus three (3) animals as control) having a body weight of 24±3 g were used.

Surgical Method:

23 black C57BL/6 mice were randomly divided into four groups; (I) a wound healing-improving material group with colon anastomization (n=10), (II) a wound healing-improving material control group without colon anastomization (n=2), (III) an NaCl (0.9%) control group with colon anastomization (n=10), and (IV) an NaCl (0.9%) control group without colon anatomization (n=1).

The mice were anesthetized with carprofen (5 mg/kg body weight) via subcutaneous injection. After anesthetic induction, the animals were fixed in the supine position, and the coat was shaved off. After disinfection (sterile washing) the abdomen was masked. The abdominal cavity was then opened, layer by layer, over 1.5 cm by median laparotomy, and the intestine was exposed. The cecum was carefully eventrated, and the colon was transected approximately 1 cm distal to the cecum. The colon anastomosis was subsequently created using twelve (12) single-button sutures (Vicryl 8/0) under 16× magnification.

The wound healing-improving material was immediately applied as a powder. The wound healing-improving material applied to the colon anastomosis was subsequently transformed from a powder into a gel, using 0.9% sterile saline solution.

In the two NaCl (0.9%) control groups, the same quantity of 0.9% sterile saline solution was applied instead of the wound healing-improving material.

No colon transection with subsequent colon anastomosis was performed in the two control groups without colon anastomization. The wound healing-improving material or 0.9% sterile saline solution was respectively provided, directly after laparotomy, to the intact colon approximately 1 cm distal to the cecum.

After conclusion of the respective surgical procedures, the abdominal cavity was closed by a double-row continuous suture with absorbable 4/0 thread. The wound was aseptically covered. The mice were returned to their cages and tended to for seven (7) days. All animals received a subcutaneous injection of Meloxican (sic: Meloxicam) (1 mg/kg body weight) as pain medication, as well as a subcutaneous injection of 5 mL isotone saline solution to avoid dehydration. Tramadol (2.5 mg/100 mL drinking water) was added to the drinking water for further postoperative analgesia.

Seven (7) days after the surgery, the animals were euthanized by CO2 asphyxiation. Relaparotomy was performed, and the strength of the anastomosis was measured by the decrease in the anastomotic bursting pressure. For this purpose, the anastomosis-bearing portion of the colon was removed and inserted into a measuring apparatus. The intraluminal pressure was increased until the anastomosis burst, and the pressure difference was measured. In addition, tissue samples were taken, and histological hematoxylin-eosin dye analysis was carried out to verify the wound healing.

Statistical Analysis:

The data were calculated and analyzed using the Graphpad Prism 7. A Welch t-test for two independent random samples with unequal standard deviations in both populations was used as the Significance Test.

Experimental results:

Seven (7) days after the anastomization, the value for the anastomotic bursting pressure in the NaCl (0.9%) control group with colon anastomization was significantly lower than in the wound healing-improving material group with colon anastomization (t=3.0; p<0.05). There were no significant differences in the wound healing-improving material group with colon anastomization compared to the wound healing-improving material control group without colon anastomization (t=2.5; p>0.05), or compared to the NaCl (0.9%) control group without colon anastomization.

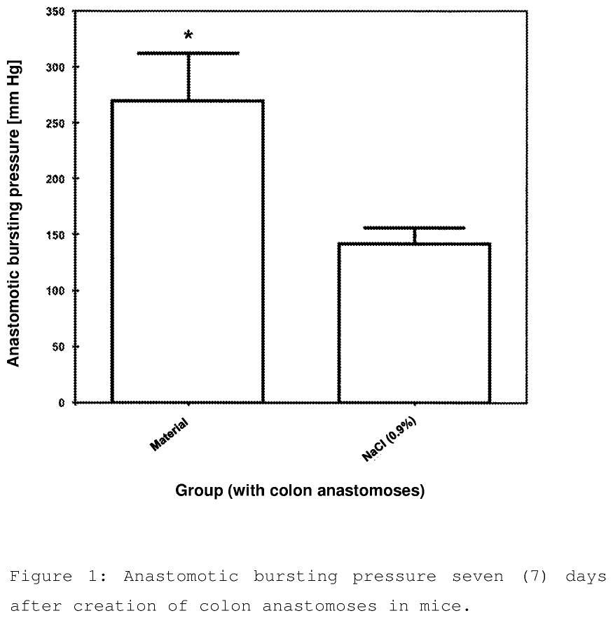

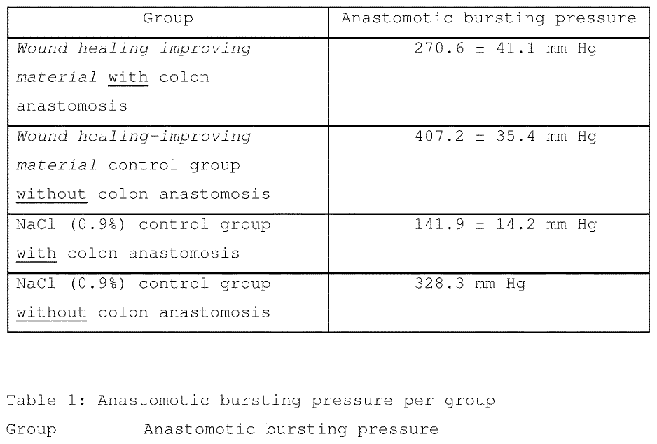

The results are summarized in FIG. 1 and Table 1.

1. A method for preparing a medicament for treating anastomoses, the method for preparing including:
a) providing a carboxymethylated starch; or
b) providing a carboxymethylated starch and an epichlorohydrin-modified starch; or
c) providing an epichlorohydrin-modified starch.

2. Use of a medicament for treating anastomoses in and/or on the intestine, the medicament for treating anastomoses including:
a) a carboxymethylated starch; or
b) a carboxymethylated starch and epichlorohydrin-modified starch; or
c) an epichlorohydrin-modified starch.

3. Use of:
a) carboxymethylated starch; and/or
b) epichlorohydrin-modified starch
for preparing a medicament for treating anastomoses.

4. Use of the medicament according to claim 3 for treating anastomoses of mesenchymal organs.

5. Use of the medicament according to claim 4, wherein:
a) the mesenchymal organs are bones, tendons, muscles, fat tissue, and connective tissue.

6. Use of the medicament according to claim 3, wherein:
a) the medicament is topically applied.

7. Use of the medicament according to claim 3, wherein:
a) the medicament is for treating epithelial defects.

8. Use of the medicament according to claim 3, wherein:
a) the medicament is for treating wounds.

9. Use of the medicament according to claim 3, wherein:
a) the medicament is in a gel.

10. The method for preparing a medicament according to claim 1, wherein:
a) the medicament further contains water and/or at least one salt.

11. The method for preparing a medicament according to claim 1, wherein:
a) the carboxymethylated starch and/or the epichlorohydrin-modified starch is in the form of particles having a molecular weight of 500,000 to 11,000,000 Daltons and/or the starch has a particle diameter of 30 μm to 100 μm.
